(19) United States
(12) Patent Application Publication
(10) Pub. No.: US 2023/0368912 A1
(43) Pub. Date: Nov. 16, 2023

(54) INFORMATION PROCESSING APPARATUS, NON-TRANSITORY COMPUTER READABLE MEDIUM, AND INFORMATION PROCESSING METHOD

(71) Applicant: TOYOTA JIDOSHA KABUSHIKI KAISHA, Toyota-shi, Aichi-ken (JP)

(72) Inventors: Yoshikazu JIKUHARA, Miyoshi-shi (JP); Seii SAI, Yokohama-shi (JP); Ibuki SHIMADA, Miyoshi-shi (JP); Takahiro AOKI, Saitama-shi (JP); Keishi KINOSHITA, Setagaya-ku (JP)

(21) Appl. No.: 18/309,860

(22) Filed: May 1, 2023

(30) Foreign Application Priority Data
May 2, 2022 (JP) 2022-076337

(51) Int. Cl. G16H 40/67; G16H 80/00; G16H 10/60; B60W 60/00

(52) U.S. Cl. CPC G16H 40/67; B60W 60/00253; G16H 10/60; G16H 80/00

(57) ABSTRACT

An information processing apparatus includes a controller and a memory. The information processing apparatus is communicable with a patient-side terminal and doctor-side terminals via a network. The controller is configured to execute operations including: receiving, from the patient-side terminal, measurement data on a patient in a medical checkup; identifying an available doctor in the memory; transmitting the measurement data to a doctor-side terminal corresponding to the doctor; receiving, from the doctor-side terminal, a diagnostic result for the measurement data; and transmitting the diagnostic result to the patient-side terminal.

### CROSS-REFERENCE TO RELATED APPLICATION

This application claims priority to Japanese Patent Application No. 2022-076337, filed on May 2, 2022, the entire contents of which are incorporated herein by reference.

### TECHNICAL FIELD

The present disclosure relates to an information processing apparatus, a program, and an information processing method.

### BACKGROUND

A server that determines medical checkup equipment to be installed in a vehicle, based on information on a user who plans to receive a medical checkup, is known (for example, Patent Literature (PTL) 1).

### Citation List

### Patent Literature

PTL 1: JP 2021-022332 A

### SUMMARY

A doctor who diagnoses measurement data on a patient in a medical checkup may be difficult to secure. In this case, it takes long time for the patient to know a result of the medical checkup, owing to time required to secure the doctor.

It would be helpful to assist a patient to early grasp a result of a medical checkup.

An information processing apparatus according to an embodiment of the present disclosure is an information processing apparatus including a controller and a memory, the information processing apparatus communicable with a patient-side terminal and doctor-side terminals via a network, the controller configured to execute operations including:
receiving, from the patient-side terminal, measurement data on a patient in a medical checkup;
identifying an available doctor in the memory;
transmitting the measurement data to a doctor-side terminal corresponding to the doctor;
receiving, from the doctor-side terminal, a diagnostic result for the measurement data; and
transmitting the diagnostic result to the patient-side terminal.

A program according to an embodiment of the present disclosure is configured to cause a computer, as an information processing apparatus including a controller and a memory, the information processing apparatus communicable with a patient-side terminal and doctor-side terminals via a network, to execute operations, the operations including:
receiving, from the patient-side terminal, measurement data on a patient in a medical checkup;
identifying an available doctor in the memory;
transmitting the measurement data to a doctor-side terminal corresponding to the doctor;
receiving, from the doctor-side terminal, a diagnostic result for the measurement data; and
transmitting the diagnostic result to the patient-side terminal.

An information processing method according to an embodiment of the present disclosure is an information processing method performed by an information processing apparatus including a controller and a memory, the information processing apparatus communicable with a patient-side terminal and doctor-side terminals via a network, the information processing method including:
receiving, from the patient-side terminal, measurement data on a patient in a medical checkup;
identifying an available doctor in the memory;
transmitting the measurement data to a doctor-side terminal corresponding to the doctor;
receiving, from the doctor-side terminal, a diagnostic result for the measurement data; and
transmitting the diagnostic result to the patient-side terminal.

According to an embodiment of the present disclosure, it is possible to assist a patient to early grasp a result of a medical checkup.

### BRIEF DESCRIPTION OF THE DRAWINGS

In the accompanying drawings:

FIG. 1 is a schematic diagram of an information processing system according to the present embodiment;

FIG. 2 is a block diagram illustrating a configuration of an information processing apparatus;

FIG. 3 is a block diagram illustrating a configuration of a patient-side terminal;

FIG. 4 is a block diagram illustrating a configuration of a doctor-side terminal;

FIG. 5 is a diagram illustrating a data structure of an availability status database (DB);

FIG. 6 is a diagram illustrating a data structure of a diagnostic object DB; and

FIG. 7 is a flowchart illustrating operations of the information processing apparatus.

### DETAILED DESCRIPTION

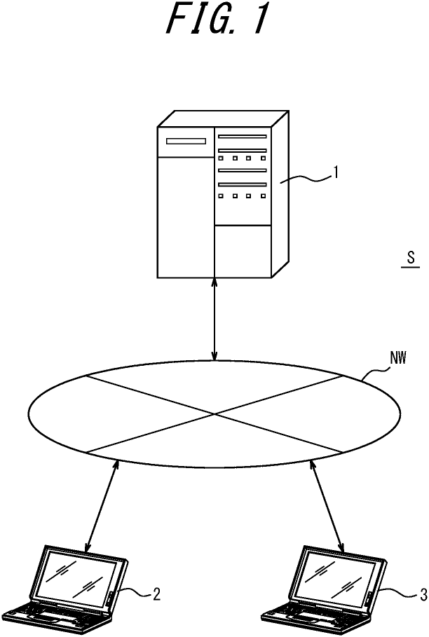

FIG. 1 is a schematic diagram of an information processing system S according to the present embodiment. The information processing system S includes an information processing apparatus 1, a patient-side terminal 2, and a doctor-side terminal 3. The information processing apparatus 1 can communicate with the patient-side terminal 2 and the doctor-side terminal 3 via a network NW. The network NW is a wired network or a wireless network and includes, for example, a mobile communication network or the Internet.

FIG. 1 illustrates one each of the information processing apparatus 1, the patient-side terminal 2, and the doctor-side terminal 3 for convenience of explanation. However, the number of information processing apparatuses 1, the number of patient-side terminals 2, and the number of doctor-side terminals 3 are not limited to this. For example, the processing to be executed by the information processing apparatus 1 may be executed by a plurality of distributed information processing apparatuses 1.

An outline of processing executed by the information processing apparatus 1 of the present embodiment will be described. A controller 11 of the information processing apparatus 1 is configured to execute operations including: receiving, from the patient-side terminal 2, measurement data on a patient in a medical checkup; identifying an available doctor; transmitting the measurement data to the doctor-side terminal 3, which corresponds to the doctor; receiving, from the doctor-side terminal 3, a diagnostic result for the measurement data; and transmitting the diagnostic result to the patient-side terminal 2. With this configuration, the information processing apparatus 1 enables the patient who has received the medical checkup to obtain the diagnostic result from the doctor online, thereby assisting the patient to early grasp the result of the medical checkup.

The information processing apparatus 1 may be a server that assists in providing the service. The information processing apparatus 1 may be, for example, installed in a facility dedicated to a provider or in a shared facility including a data center.

The patient-side terminal 2 is a terminal operated by an entity performing a medical checkup or by a patient receiving the medical checkup. The doctor-side terminal 3 is a terminal operated by a doctor or medical personnel who diagnoses measurement data of the medical checkup. Each of the patient-side terminal 2 and the doctor-side terminal 3 may be, for example, a general-purpose device such as a PC or a dedicated device. The term “PC” is an abbreviation of personal computer. As an alternative example, the patient-side terminal 2 and the doctor-side terminal 3 are each mobile devices such as mobile phones, smartphones, wearable devices, or tablets.

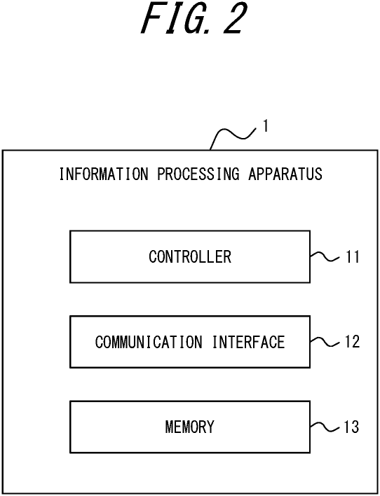

With reference to FIG. 2, an internal configuration of the information processing apparatus 1 will be described in detail.

The information processing apparatus 1 includes the controller 11, a communication interface 12, and a memory 13. The components of the information processing apparatus 1 are communicably connected to one another via, for example, dedicated lines.

The controller 11 includes, for example, one or more general purpose processors including a Central Processing Unit (CPU) or a Micro Processing Unit (MPU). The controller 11 may include one or more dedicated processors that are dedicated to specific processing. The controller 11 may include one or more dedicated circuits instead of the processors. Examples of dedicated circuits may include a Field-Programmable Gate Array (FPGA) and an Application Specific Integrated Circuit (ASIC). The controller 11 may include an Electronic Control Unit (ECU).

The communication interface 12 includes, for connecting to a network, one or more communication modules that conform to wired or wireless Local Area Network (LAN) standards. The communication interface 12 may include a module conforming to one or more mobile communication standards including the Long Term Evolution (LTE) standard, the 4th Generation (4G) standard, or the 5th Generation (5G) standard. The communication interface 12 may include one or more communication modules conforming to near field communication standards or specifications, including Bluetooth^{®} (Bluetooth is a registered trademark in Japan, other countries, or both), AirDrop^{®} (AirDrop is a registered trademark in Japan, other countries, or both), IrDA, ZigBee^{®} (ZigBee is a registered trademark in Japan, other countries, or both), Felica^{®} (Felica is a registered trademark in Japan, other countries, or both), or RFID. The communication interface 12 transmits and receives any information via the network.

The memory 13 includes, for example, a semiconductor memory, a magnetic memory, an optical memory, or a combination of at least two of these, but is not limited to these. The semiconductor memory is, for example, RAM or ROM. The RAM is, for example, SRAM or DRAM. The ROM is, for example, EEPROM. The memory 13 may function as, for example, a main memory, an auxiliary memory, or a cache memory. The memory 13 may store information resulting from analysis or processing performed by the controller 11. The memory 13 may store various types of information and the like regarding operations or control of the information processing apparatus 1. The memory 13 may store a system program, an application program, embedded software, and the like. The memory 13 may be provided outside the information processing apparatus 1 and accessed by the information processing apparatus 1. The memory 13 may include an availability status DB and a diagnostic object DB, which are described later.

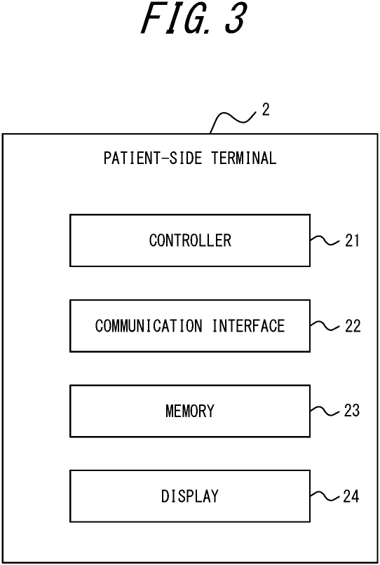

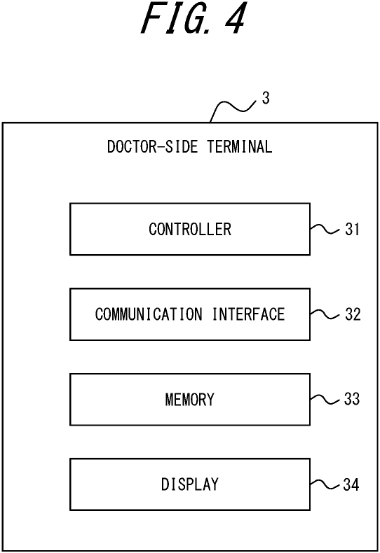

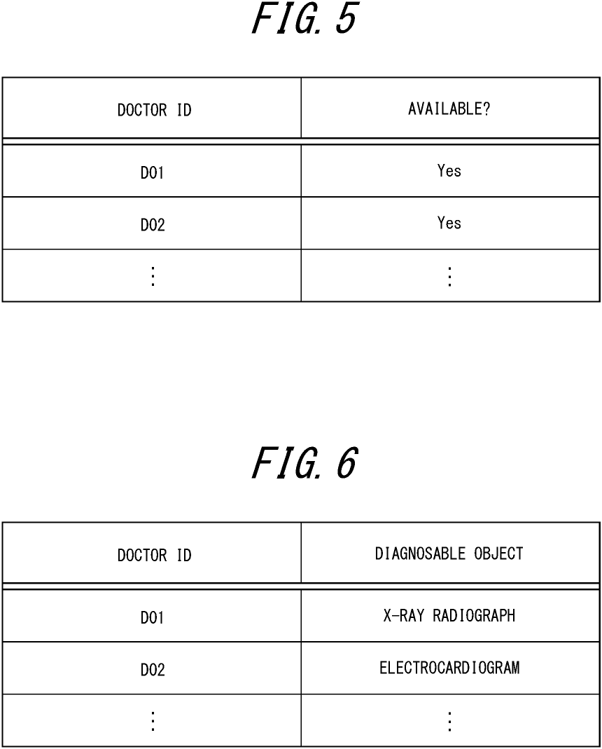

With reference to FIG. 3, an internal configuration of the patient-side terminal 2 will be described in detail.

The patient-side terminal 2 includes a controller 21, a communication interface 22, a memory 23, and a display 24. The components of the patient-side terminal 2 are communicably connected to one another, for example, via dedicated lines.

The hardware configurations of the controller 21, the communication interface 22, and the memory 23 of the patient-side terminal 2 may be the same as those of the controller 11, the communication interface 12, and the memory 13 of the information processing apparatus 1, respectively. An explanation here is omitted.

The display 24 is, for example, a display. The display is, for example, an LCD or an organic EL display. The term “LCD” is an abbreviation of liquid crystal display. The term “EL” is an abbreviation of electro luminescence. The display 24, instead of being included in the patient-side terminal 2, may be connected to the patient-side terminal 2 as an external output device. As the connection method, any technology such as USB, HDMI^{®}, or Bluetooth^{®} can be used.

With reference to FIG. 4, an internal configuration of the doctor-side terminal 3 will be described in detail.

The doctor-side terminal 3 includes a controller 31, a communication interface 32, a memory 33, and a display 34. The components of the doctor-side terminal 3 are communicably connected to one another, for example, via dedicated lines.

The hardware configurations of the controller 31, the communication interface 32, the memory 33, and the display 34 of the doctor-side terminal 3 are the same as those of the controller 21, the communication interface 22, the memory 23, and the display 24 of the patient-side terminal 2, respectively. An explanation here is omitted.

Hereinafter, processing executed in the information processing system S according to the present embodiment will be described in detail. As an example here, the patient-side terminal 2 is installed in a site of a medical checkup. The doctor-side terminals 3 are installed in different locations (e.g., medical institutions) from the site of the medical checkup.

Upon accepting an input of measurement data on a patient of the medical checkup, the controller 21 of the patient-side terminal 2 controls the communication interface 22 to transmit the measurement data to the information processing apparatus 1. The measurement data in the present embodiment is data that allows a doctor to relatively early present a diagnostic result online, such as data measured by X-ray or electrocardiogram. However, as an alternative example, the measurement data may include data that requires relatively long time to be examined and diagnosed, such as data measured by blood tests.

When the communication interface 12 receives the measurement data, the controller 11 of the information processing apparatus 1 refers to the memory 13. The controller 11 refers to an availability status DB illustrated in FIG. 5 and identifies available doctors (in this case, doctors D01 and D02). In the availability status DB, for example, the availability status of each of nationwide doctors is registered and updated as appropriate.

As an additional example, the controller 11 may determine a diagnostic object from the measurement data. The diagnostic object is, for example, an X-ray radiograph, an electrocardiogram, or the like. The controller 11 may refer to a diagnostic object DB illustrated in FIG. 6 and identify an available doctor who is able to diagnose the diagnostic object. For example, when the diagnostic object of the measurement data is an X-ray radiograph, the controller 11 identifies a doctor D01 who is able to diagnose the X-ray radiograph.

As an alternative example, when identifying a doctor, the controller 11 may inquire of the doctor-side terminals 3 of all doctors registered in the availability status DB or available doctors whether the doctors are able to diagnose the measurement data. The controller 11 identifies a doctor who has responded that he/she is able to diagnose the measurement data. Thus, so-called “job matching” may take place.

The controller 11 controls the communication interface 12 to transmit the measurement data to a doctor-side terminal 3 corresponding to the identified doctor. As an additional example, the controller 11 may control the communication interface 12 to transmit an electronic medical record, which is received from a terminal operated by a family doctor of the patient, in addition to the measurement data. The timing of transmission of the electronic medical record and the timing of transmission of the measurement data may be simultaneous or different.

Upon receiving the measurement data, the doctor-side terminal 3 prompts the doctor or the like to input a diagnostic result for the measurement data. The diagnostic result may include, for example, information indicating the presence or absence of necessity to transport the patient to a hospital. The necessity of transport may be determined from the presence or absence of concerns, the presence or absence of necessity of re-examination, or the like.

Upon receiving an input of diagnostic result data, the doctor-side terminal 3 transmits the diagnostic result to the information processing apparatus 1. Upon receiving the diagnostic result, the information processing apparatus 1 transmits the diagnostic result to the patient-side terminal 2. The controller 21 of the patient-side terminal 2 controls the display 24 to display the diagnostic result. The patient can see the diagnostic result.

As an additional example, when the diagnostic result indicates the necessity to transport the patient, the controller 11 may determine to transport the patient by a vehicle to an affiliated hospital on the day of the medical checkup or later. For example, the controller 11 may command an automated vehicle, which can communicate via the network NW, to transport the patient to the hospital. Information on a date and time of the transport, a location to pick up the patient, a transport route, the hospital as a destination, and the like may be input by the patient or the affiliated hospital.

As an alternative example, the controller 11 can transmit the measurement data to multiple doctor-side terminals 3 corresponding to multiple available doctors, and receive diagnosis results from the multiple doctor-side terminals 3.

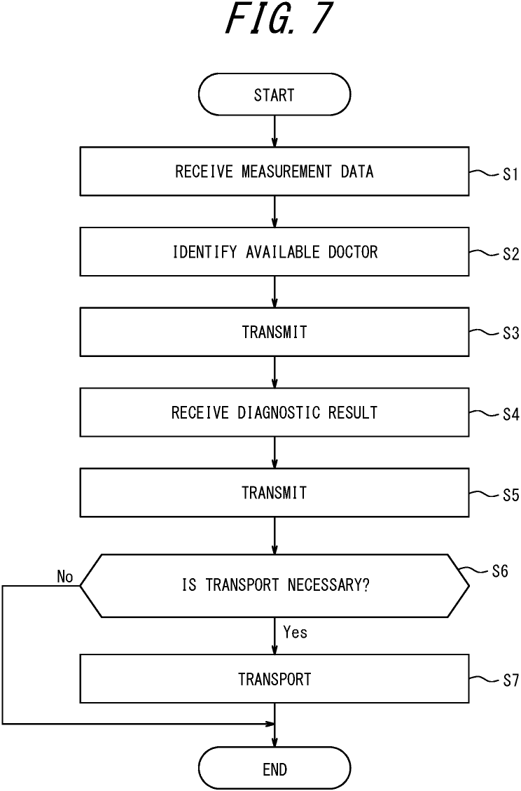

An information processing method performed by the controller 11 of the information processing apparatus 1 will be described with reference to FIG. 7.

In step S1, the controller 11 receives measurement data from the patient-side terminal 2.

In step S2, the controller 11 identifies an available doctor in the memory 13. As an additional example, the controller 11 may determine a diagnostic object from the measurement data, and identify an available doctor who is able to diagnose the diagnostic object.

In step S3, the controller 11 transmits the measurement data to the doctor-side terminal 3 corresponding to the identified doctor.

In step S4, the controller 11 receives a diagnostic result from the doctor-side terminal 3.

In step S5, the controller 11 transmits the diagnostic result to the patient-side terminal 2.

In step S6, the controller 11 determines whether the patient needs to be transported. When Yes in step S6, the controller 11 commands an automated vehicle to transport the patient to a hospital in step S7. Steps S6 and S7 are optional.

According to the present embodiment, as described above, the controller 11 of the information processing apparatus 1 is configured to execute operations including: receiving, from the patient-side terminal 2, measurement data on a patient in a medical checkup; identifying an available doctor; transmitting the measurement data to a doctor-side terminal 3 corresponding to the doctor; receiving, from the doctor-side terminal 3, a diagnostic result for the measurement data; and transmitting the diagnostic result to the patient-side terminal 2. With this configuration, the information processing apparatus 1 enables the patient who has received the medical checkup to obtain the diagnostic result from the doctor online, thereby assisting the patient to early grasp the result of the medical checkup.

According to the present embodiment, the operations of the controller 11 include: identifying multiple available doctors; transmitting the measurement data to multiple doctor-side terminals 3 corresponding to the multiple doctors; and receiving diagnostic results from the multiple doctor-side terminals 3. According to this configuration, the information processing apparatus 1 allows the patient to be double-checked or obtain a second opinion, thus enhancing a sense of relief of the patient.

According to the present embodiment, the diagnostic result includes information indicating the presence or absence of necessity to transport the patient to a hospital. Further, when the diagnostic result indicates that the patient needs to be transported to a hospital, the operations of the controller 11 includes commanding an automated vehicle via the network to transport the patient to the hospital. According to this configuration, the information processing apparatus 1 facilitates letting the patient with a health problem take medical treatment as soon as possible.

According to the present embodiment, the operations of the controller 11 include: receiving an electronic medical record of the patient from a terminal operated by a family doctor of the patient; and transmitting the electronic medical record, in addition to the measurement data, to the doctor-side terminal 3. According to this configuration, the information processing apparatus 1 can assist the doctor to make a diagnosis more appropriately.

According to the present embodiment, the operations of the controller 11 includes: determining a diagnostic object from the measurement data; identifying, in the memory 13, an available doctor who is able to diagnose the diagnostic object; and transmitting the measurement data to a doctor-side terminal 3 corresponding to the identified doctor. This configuration allows the information processing apparatus 1 to request a diagnosis to an appropriate doctor who is able to diagnose the diagnostic object.

According to the present embodiment, the operations of the controller 11 include: inquiring of one or more doctor-side terminals 3 corresponding to one or more doctors whether the doctors are able to diagnose the measurement data; and transmitting the measurement data to a doctor-side terminal 3 corresponding to a doctor who is able to diagnose the measurement data. This configuration allows the information processing apparatus 1 to more easily identify the doctor who is able to diagnose the measurement data.

While the present disclosure has been described with reference to the drawings and examples, it should be noted that various modifications and revisions may be implemented by those skilled in the art based on the present disclosure. Other modifications can be made without departing from the spirit of the present disclosure. For example, functions or the like included in each means or each step can be rearranged without logical inconsistency, and a plurality of means or steps can be combined into one or divided.

For example, in the aforementioned embodiment, a program configured to execute all or some of the functions or processing of the information processing apparatus 1 can be recorded on a computer readable recording medium. The computer readable recording medium includes a non-transitory computer readable medium and is, for example, a magnetic recording apparatus, an optical disc, a magnetooptical recording medium, or a semiconductor memory. The program is distributed, for example, by selling, transferring, or lending a portable recording medium such as a Digital Versatile Disc (DVD) or a Compact Disc Read Only Memory (CD-ROM) on which the program is recorded. The program may also be distributed by storing the program in a storage of any server and transmitting the program from any server to another computer. The program may be provided as a program product. The present disclosure can also be implemented as a program executable by a processor.

The computer temporarily stores in a main memory, for example, a program recorded on a portable recording medium, or a program transferred from the server. Then, the computer reads the program stored in the main memory using a processor, and executes processes in accordance with the read program using the processor. The computer may read a program directly from the portable recording medium, and execute processes in accordance with the program. The computer may, each time a program is transferred from the server to the computer, sequentially execute processes in accordance with the received program. Instead of transferring a program from the server to the computer, processes may be executed by a so-called ASP type service that realizes functions only by execution instructions and result acquisitions. The term “ASP” is an abbreviation of application service provider. Programs encompass information that is to be used for processing by an electronic computer and is thus equivalent to a program. For example, data that is not a direct command to a computer but has a property that regulates processing of the computer is “equivalent to a program” in this context.

A part of the embodiment of the present disclosure will be exemplarily described below. However, it should be noted that the embodiment of the present disclosure is not limited to these.

### Appendix 1

An information processing apparatus comprising a controller and a memory, the information processing apparatus communicable with a patient-side terminal and doctor-side terminals via a network, the controller configured to execute operations comprising:
receiving, from the patient-side terminal, measurement data on a patient in a medical checkup;
identifying an available doctor in the memory;
transmitting the measurement data to a doctor-side terminal corresponding to the doctor;
receiving, from the doctor-side terminal, a diagnostic result for the measurement data; and
transmitting the diagnostic result to the patient-side terminal.

### Appendix 2

The information processing apparatus according to appendix 1, wherein the operations comprise:
identifying a plurality of available doctors in the memory;
transmitting the measurement data to a plurality of doctor-side terminals corresponding to the plurality of doctors; and
receiving diagnostic results from the plurality of doctor-side terminals.

### Appendix 3

The information processing apparatus according to appendix 1 or 2, wherein the diagnostic result includes information indicating presence or absence of necessity to transport the patient to a hospital.

### Appendix 4

The information processing apparatus according to appendix 3, wherein the operations comprise commanding an automated vehicle via the network to transport the patient to the hospital, when the diagnostic result indicates that the patient needs to be transported to the hospital.

### Appendix 5

The information processing apparatus according to any one of appendixes 1 to 4, wherein the operations comprise:
receiving an electronic medical record of the patient from a terminal operated by a family doctor of the patient; and
transmitting the electronic medical record, in addition to the measurement data, to the doctor-side terminal.

### Appendix 6

The information processing apparatus according to any one of appendixes 1 to 5, wherein the operations comprise:
determining a diagnostic object from the measurement data;
identifying, in the memory, an available doctor who is able to diagnose the diagnostic object; and
transmitting the measurement data to a doctor-side terminal corresponding to the identified doctor.

### Appendix 7

The information processing apparatus according to any one of appendixes 1 to 6, wherein the operations comprise:
inquiring one or more doctor-side terminals corresponding to one or more doctors whether the doctors are able to diagnose the measurement data; and
transmitting the measurement data to a doctor-side terminal corresponding to a doctor who is able to diagnose the measurement data.

### Appendix 8

A program configured to cause a computer, as an information processing apparatus including a controller and a memory, the information processing apparatus communicable with a patient-side terminal and doctor-side terminals via a network, to execute operations, the operations including:
receiving, from the patient-side terminal, measurement data on a patient in a medical checkup;
identifying an available doctor in the memory;
transmitting the measurement data to a doctor-side terminal corresponding to the doctor;
receiving, from the doctor-side terminal, a diagnostic result for the measurement data; and
transmitting the diagnostic result to the patient-side terminal.

### Appendix 9

The program according to appendix 8, wherein the operations comprise:
identifying a plurality of available doctors in the memory;
transmitting the measurement data to a plurality of doctor-side terminals corresponding to the plurality of doctors; and
receiving diagnostic results from the plurality of doctor-side terminals.

### Appendix 10

The program according to appendix 8 or 9, wherein the diagnostic result includes information indicating presence or absence of necessity to transport the patient to a hospital.

### Appendix 11

The program according to appendix 10, wherein the operations comprise commanding an automated vehicle via the network to transport the patient to the hospital, when the diagnostic result indicates that the patient needs to be transported to the hospital.

### Appendix 12

The program according to any one of appendixes 8 to 11, wherein the operations comprise:
receiving an electronic medical record of the patient from a terminal operated by a family doctor of the patient; and
transmitting the electronic medical record, in addition to the measurement data, to the doctor-side terminal.

### Appendix 13

The program according to any one of appendixes 8 to 12, wherein the operations comprise:
determining a diagnostic object from the measurement data;
identifying, in the memory, an available doctor who is able to diagnose the diagnostic object; and
transmitting the measurement data to a doctor-side terminal corresponding to the identified doctor.

### Appendix 14

The program according to any one of appendixes 8 to 13, wherein the operations comprise:
inquiring one or more doctor-side terminals corresponding to one or more doctors whether the doctors are able to diagnose the
measurement data; and
transmitting the measurement data to a doctor-side terminal corresponding to a doctor who is able to diagnose the measurement data.

### Appendix 15

An information processing method performed by an information processing apparatus including a controller and a memory, the information processing apparatus communicable with a patient-side terminal and doctor-side terminals via a network, the information processing method comprising:
receiving, from the patient-side terminal, measurement data on a patient in a medical checkup;
identifying an available doctor in the memory;
transmitting the measurement data to a doctor-side terminal corresponding to the doctor;
receiving, from the doctor-side terminal, a diagnostic result for the measurement data; and
transmitting the diagnostic result to the patient-side terminal.

### Appendix 16

The information processing method according to appendix 15, comprising:
identifying a plurality of available doctors in the memory;
transmitting the measurement data to a plurality of doctor-side terminals corresponding to the plurality of doctors; and
receiving diagnostic results from the plurality of doctor-side terminals.

### Appendix 17

The information processing method according to appendix 15 or 16, wherein the diagnostic result includes information indicating presence or absence of necessity to transport the patient to a hospital.

### Appendix 18

The information processing method according to appendix 17, comprising commanding an automated vehicle via the network to transport the patient to the hospital, when the diagnostic result indicates that the patient needs to be transported to the hospital.

### Appendix 19

The information processing method according to any one of appendixes 15 to 18, comprising:
receiving an electronic medical record of the patient from a terminal operated by a family doctor of the patient; and
transmitting the electronic medical record, in addition to the measurement data, to the doctor-side terminal.

### Appendix 20

The information processing method according to any one of appendixes 15 to 19, comprising:
determining a diagnostic object from the measurement data;
identifying, in the memory, an available doctor who is able to diagnose the diagnostic object; and
transmitting the measurement data to a doctor-side terminal corresponding to the identified doctor.

1. An information processing apparatus comprising a controller and a memory, the information processing apparatus communicable with a patient-side terminal and doctor-side terminals via a network, the controller configured to execute operations comprising:
receiving, from the patient-side terminal, measurement data on a patient in a medical checkup;
identifying an available doctor in the memory;
transmitting the measurement data to a doctor-side terminal corresponding to the doctor;
receiving, from the doctor-side terminal, a diagnostic result for the measurement data; and
transmitting the diagnostic result to the patient-side terminal.

2. The information processing apparatus according to claim 1, wherein the operations comprise:
identifying a plurality of available doctors in the memory;
transmitting the measurement data to a plurality of doctor-side terminals corresponding to the plurality of doctors; and
receiving diagnostic results from the plurality of doctor-side terminals.

3. The information processing apparatus according to claim 1, wherein the diagnostic result includes information indicating presence or absence of necessity to transport the patient to a hospital.

4. The information processing apparatus according to claim 3, wherein the operations comprise commanding an automated vehicle via the network to transport the patient to the hospital, when the diagnostic result indicates that the patient needs to be transported to the hospital.

5. The information processing apparatus according to claim 1, wherein the operations comprise:
receiving an electronic medical record of the patient from a terminal operated by a family doctor of the patient; and
transmitting the electronic medical record, in addition to the measurement data, to the doctor-side terminal.

6. The information processing apparatus according to claim 1, wherein the operations comprise:
determining a diagnostic object from the measurement data;
identifying, in the memory, an available doctor who is able to diagnose the diagnostic object; and
transmitting the measurement data to a doctor-side terminal corresponding to the identified doctor.

7. The information processing apparatus according to claim 1, wherein the operations comprise:
inquiring one or more doctor-side terminals corresponding to one or more doctors whether the doctors are able to diagnose the measurement data; and
transmitting the measurement data to a doctor-side terminal corresponding to a doctor who is able to diagnose the measurement data.

8. A non-transitory computer readable medium storing a program configured to cause a computer, as an information processing apparatus including a controller and a memory, the information processing apparatus communicable with a patient-side terminal and doctor-side terminals via a network, to execute operations, the operations comprising:
receiving, from the patient-side terminal, measurement data on a patient in a medical checkup;
identifying an available doctor in the memory;
transmitting the measurement data to a doctor-side terminal corresponding to the doctor;
receiving, from the doctor-side terminal, a diagnostic result for the measurement data; and
transmitting the diagnostic result to the patient-side terminal.

9. The non-transitory computer readable medium according to claim 8, wherein the operations comprise:
identifying a plurality of available doctors in the memory;
transmitting the measurement data to a plurality of doctor-side terminals corresponding to the plurality of doctors; and
receiving diagnostic results from the plurality of doctor-side terminals.

10. The non-transitory computer readable medium according to claim 8, wherein the diagnostic result includes information indicating presence or absence of necessity to transport the patient to a hospital.

11. The non-transitory computer readable medium according to claim 10, wherein the operations comprise commanding an automated vehicle via the network to transport the patient to the hospital, when the diagnostic result indicates that the patient needs to be transported to the hospital.

12. The non-transitory computer readable medium according to claim 8, wherein the operations comprise:
receiving an electronic medical record of the patient from a terminal operated by a family doctor of the patient; and
transmitting the electronic medical record, in addition to the measurement data, to the doctor-side terminal.

13. The non-transitory computer readable medium according to claim 8, wherein the operations comprise:
determining a diagnostic object from the measurement data;
identifying, in the memory, an available doctor who is able to diagnose the diagnostic object; and
transmitting the measurement data to a doctor-side terminal corresponding to the identified doctor.

14. The non-transitory computer readable medium according to claim 8, wherein the operations comprise:
inquiring one or more doctor-side terminals corresponding to one or more doctors whether the doctors are able to diagnose the measurement data; and
transmitting the measurement data to a doctor-side terminal corresponding to a doctor who is able to diagnose the measurement data.

15. An information processing method performed by an information processing apparatus including a controller and a memory, the information processing apparatus communicable with a patient-side terminal and doctor-side terminals via a network, the information processing method comprising:
receiving, from the patient-side terminal, measurement data on a patient in a medical checkup;
identifying an available doctor in the memory;
transmitting the measurement data to a doctor-side terminal corresponding to the doctor;
receiving, from the doctor-side terminal, a diagnostic result for the measurement data; and
transmitting the diagnostic result to the patient-side terminal.

16. The information processing method according to claim 15, comprising:
identifying a plurality of available doctors in the memory;
transmitting the measurement data to a plurality of doctor-side terminals corresponding to the plurality of doctors; and
receiving diagnostic results from the plurality of doctor-side terminals.

17. The information processing method according to claim 15, wherein the diagnostic result includes information indicating presence or absence of necessity to transport the patient to a hospital.

18. The information processing method according to claim 17, comprising commanding an automated vehicle via the network to transport the patient to the hospital, when the diagnostic result indicates that the patient needs to be transported to the hospital.

19. The information processing method according to claim 15, comprising:
receiving an electronic medical record of the patient from a terminal operated by a family doctor of the patient; and
transmitting the electronic medical record, in addition to the measurement data, to the doctor-side terminal.

20. The information processing method according to claim 15, comprising:
determining a diagnostic object from the measurement data;
identifying, in the memory, an available doctor who is able to diagnose the diagnostic object; and
transmitting the measurement data to a doctor-side terminal corresponding to the identified doctor.
